(19) United States
(12) Patent Application Publication
(10) Pub. No.: US 2009/0247722 A1
(43) Pub. Date: Oct. 1, 2009

(54) CYCLIC ALKYLENE CARBONATE-DERIVED ISOCYANATE-TERMINATED PREPOLYMERS, METHOD FOR THEIR PREPARATION AND THEIR USE

(75) Inventors: Zhiping Zhou, Shanghai (CN); Hongmei Jiang, Shanghai (CN); Yide Liang, Shanghai (CN); Yuedong Zhang, Shanghai (CN)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/410,925

(22) Filed: Mar. 25, 2009

(30) Foreign Application Priority Data
Mar. 26, 2008 (CN) 200810035167.6

(51) Int. Cl. C07D 317/28; C08G 18/70

(52) U.S. Cl. 528/69; 549/229

(57) ABSTRACT

The present invention pertains to isocyanate-terminated prepolymers prepared from cyclic alkylene carbonates, the method for preparing the same and the use thereof in preparing polyurethanes, polyureas, polyurethane elastomer, and polyurethane mouldings. The incorporation of cyclic alkylene carbonates into the isocyanate-terminated prepolymers of the present invention improves their liquid stability at low temperatures while maintaining their physical properties.

### RELATED APPLICATIONS

This application claims benefit to Chinese Patent Application No. 2008 10 035 167.6, filed Mar. 6, 2008, which is incorporated herein by reference in its entirety for all useful purposes.

### BACKGROUND OF THE INVENTION

Polyurethanes are normally prepared by reacting isocyanate components, polyol components and other additives. These isocyanate components can be isocyanate-terminated prepolymers. For example, diphenylmethane diisocyanate (MDI) based prepolymers are widely used for preparing polyurethane products, such as polyurethane elastomers or moulded polyurethane products.

Isocyanate-terminated prepolymer is usually required to be liquid at normal temperatures in order to be conveniently stored and transported. However, at low temperatures, free polyisocyanate molecules may crystallize out of these liquid prepolymers, which themselves may freeze at even lower temperatures. In general, the higher the NCO content of the prepolymer, the higher its freezing point and the easier it is for the prepolymer to form crystals or be frozen. Therefore, it is less convenient to store and transport the prepolymer at low temperatures.

A variety of methods have been developed to reduce the freezing point of isocyanate-terminated prepolymers in order to improve their liquid stability. For example, EP 99116964, U.S. 2006/0128928, U.S. 2005/0101754, and U.S. Pat. No. 5,567,793 disclose that adding modified MDI (such as carbo-diimidized MDI or uretone-iminized MDI) during the preparation of MDI prepolymer can improve the liquid stability of the prepolymer. GB 2334720 discloses that adding high amounts of 2,4-MDI and 2,2-MDI during the preparation of MDI prepolymer can reduce the freezing point of the prepolymer. However, these methods sacrifice the physical properties of the prepolymer in order to reduce its freezing point. GB 2193504 and U.S. Pat. No. 4,757,095 disclose that lactones and lactams can act as freezing point depressants in MDI prepolymers and WO 85/00177 discloses that a variety of compounds with oxyalkylene groups can be used to modify the low temperature fluidity of MDI prepolymers at low temperatures. However, the high cost of lactones, lactams, and compounds with oxyalkylene groups limits their application in this field.

Therefore, it is an object of the present invention to develop an economical isocyanate-terminated prepolymer with improved liquid stability that not only facilitates its storage and transportation at low temperatures, but also maintains its physical properties.

This object is achieved by the isocyanate-terminated prepolymer using a cyclic alkylene carbonate. The incorporation of a cyclic alkylene carbonate into the isocyanate-terminated prepolymer imparts improved liquid stability to the prepolymer at low temperatures, which facilitates its storage and transportation, while also maintaining its physical properties.

### EMBODIMENTS OF THE INVENTION

An embodiment of the present invention is an isocyanate-terminated prepolymer prepared from
A) a polyisocyanate;
B) a polyol; and
C) a cyclic alkylene carbonate of formula:

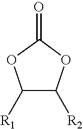

wherein
R₁ and R₂ are independently selected from the group consisting of hydrogen, linear alkyl, branched alkyl, aralkyl, naphthenic base, and aryl; and

the NCO content of said isocyanate-terminated prepolymer is in the range of from 13 to 33% by weight, based on 100% by weight of said isocyanate-terminated prepolymer.

Another embodiment of the present invention is the above isocyanate-terminated prepolymer, wherein said polyisocyanate is of formula:

R(NCO)ₙ
wherein
R is an aliphatic alkyl containing 2 to t8 carbon atoms, an aryl containing from 6 to 15 carbon atoms, or an araliphatic alkyl containing from 8 to 15 carbon atoms; and
n is an integer from 2 to 4.

Another embodiment of the present invention is the above isocyanate-terminated prepolymer, wherein said polyisocyanate is selected from the group consisting of ethylene diisocyanate; 1,4-tetramethylene diisocyanate; 1,6-hexamethylene diisocyanate; 1,2-dodecane diisocyanate; cyclobutane-1,3-diisocyanate; cyclohexane-1,3-diisocyanates; cyclohexane-1,4-diisocyanates; 1-isocyanato-3,3,5-trimethyl-5-isocyanatomethyl-cyclohexane; 2,4-hexahydrotoluene diisocyanates; 2,6-hexahydrotoluene diisocyanates; hexahydro-1,3-phenylene diisocyanate; hexahydro-1,4-phenylene diisocyanate; perhydro-2,4-diphenylmethane diisocyanate; perhydro-4,4′-diphenylmethane diisocyanate; 1,3-phenylene diisocyanate; 1,4-phenylene diisocyanate; 1,4-durol diisocyanate; 1,4-stilbene diisocyanate; 3,3′-dimethyl-4,4′-biphenylene diisocyanate, toluene 2,4-diisocyanates, toluene 2,6-diisocyanates, diphenylmethane-2,4′-diisocyanates, diphenylmethane-2,2′-diisocyanates, diphenylmethane-4,4′-diisocyanates, naphthylene-1,5-diisocyanate, and isomers and mixtures thereof.

Another embodiment of the present invention is the above isocyanate-terminated prepolymer, wherein said polyol is a polyester polyol, a polyether polyol, a polycarbonate polyol, or a mixture thereof.

Another embodiment of the present invention is the above isocyanate-terminated prepolymer, wherein said polyester polyol is prepared from the reaction of a dicarboxylic acid selected from the group consisting of succinic acid, malonic acid, glutaric acid, adipic acid, suberic acid, azelaic acid, sebacic acid, decane-dicarboxylic acid, maleic acid, fumaric acid, phthalic acid, isophthalic acid, terephthalic acid, and mixtures thereof or a dicarboxylic acid anhydride selected from the group consisting of phthalic anhydride, terachlorophthalic anhydride, maleic anhydride, and mixtures thereof with a polyhydric alcohol selected from the group consisting of ethanediol, diethylene glycol, 1,2-propanediol, 1,3-propanediol, dipropylene glycol, 1,3-methylpropanediol, 1,4-butanediol, 1,5-pentanediol, 1,6-hexanediol, neopentyl glycol, 1,10-decanediol, glycerol, trimethylol-propane, and mixtures thereof.

Another embodiment of the present invention is the above isocyanate-terminated prepolymer, wherein said polyester polyol is prepared using ε-caprolactone.

Another embodiment of the present invention is the above isocyanate-terminated prepolymer, wherein said polyether polyol is prepared from the reaction of an alkene oxide selected from the group consisting of tetrahydrofuran, ethylene oxide, 1,2-propylene oxide, 1,2-butylene oxide, 2,3-butylene oxide, styrene oxide, and mixtures thereof with a polyhydric alcohol starter selected from the group consisting of water, ethylene glycol, 1,2-propanediols, 1,3-propanediols, 1,4-butanediol, diethylene glycol, trimethylol-propane, and mixtures thereof.

Another embodiment of the present invention is the above isocyanate-terminated prepolymer, wherein said polycarbonate polyol is a polycarbonate diol prepared from the reaction of a diol selected from the group consisting of 1,2-propanediol, 1,3-propanediol, 1,4-butanediol, 1,5-pentanediol, 1,6-hexanediol, diethylene glycol, trioxyethylene glycol, and mixtures thereof with diphenyl carbonate or phosgene.

Another embodiment of the present invention is the above isocyanate-terminated prepolymer, wherein said prepolymer is further prepared from a lactone, an oxalate, or a mixture thereof.

Another embodiment of the present invention is the above isocyanate-terminated prepolymer, wherein said lactone is selected from the group consisting of γ-butyrolactone, γ-valerolactone, ε-caprolactone, αγ-dimethyl butyrolactone, βγ-dimethyl butyrolactone, γγ-dimethyl butyrolactone, and α-ethyl-γ-methyl butyrolactone, and said oxalate is selected from the group consisting of dimethyl oxalate, diethyl oxalate, and dibutyl oxalate.

Another embodiment of the present invention is the above isocyanate-terminated prepolymer, wherein the NCO content of said isocyanate-terminated prepolymer is in the range of from 13 to 24% by weight, based on 100% by weight of said isocyanate-terminated prepolymer.

Another embodiment of the present invention is the above isocyanate-terminated prepolymer, wherein the NCO content of said isocyanate-terminated prepolymer is in the range of from 15 to 20% by weight, based on 100% by weight of said isocyanate-terminated prepolymer.

Yet another embodiment of the present invention is a process for preparing the above isocyanate-terminated prepolymer comprising reacting
A) a polyisocyanate;
B) a polyol; and
C) a cyclic alkylene carbonate of formula:

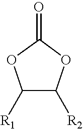

wherein
R₁ and R₂ are independently selected from the group consisting of hydrogen, linear alkyl, branched alkyl, aralkyl, naphthenic base, and aryl

wherein said cyclic alkylene carbonate is added to the reaction with A) and B), during the reaction of A) and B), or after the reaction of A) and B); and

wherein the NCO content of said isocyanate-terminated prepolymer is in the range of from 13 to 33% by weight, based on 100% by weight of said isocyanate-terminated prepolymer.

Yet another embodiment of the present invention is a polyurethane or polyurea or polyurethane elastomer or polyurethane moulding comprising the above isocyanate-terminated prepolymer.

Yet another embodiment of the present invention is a shoe sole comprising the above isocyanate-terminated prepolymer.

### DESCRIPTION OF THE INVENTION

According to the present invention, isocyanate-terminated prepolymers are prepared from (A) a polyisocyanate, (B) a polyol, and (C) a cyclic alkylene carbonate. The cyclic alkylene carbonate imparts improved liquid stability to the prepolymer at low temperatures. These cyclic alkylene carbonates have the general formula:

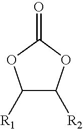

wherein R₁ and R₂ are independently selected from the group consisting of hydrogen atom, linear alkyl, branched alkyl, aralkyl, naphthenic base, and aryl.

Examples of cyclic alkylene carbonates useful for purposes of the present invention include, but are not limited to, ethylene carbonate, propylene carbonate, 1,2-butylene carbonate, 2,3-butylene carbonate, 1,2-cyclohexene carbonate, styrene carbonate, and mixtures thereof. The cyclic alkylene carbonate can be added in an amount in the range of from 1 to 15 weight %, preferably from 3 to 7 weight %, based on 100% by weight of the isocyanate-terminated prepolymer and polyols.

The cyclic alkylene carbonate can be added at any time during the preparation of the isocyanate-terminated prepolymer. For example, it can be added with the reactants, such as with the polyisocyanate component or with the polyol component. It can also be added during the reaction of the polyisocyanate component with the polyol component. It can also be added into the reaction product of the polyisocyanate component and the polyol component (e.g., once the reaction between the polyisocyanate component and the polyol component is complete).

The polyisocyanate used to prepare the isocyanate-terminated prepolymer of the present invention can be one polyisocyanate or the mixture of polyisocyanates. The polyisocyanate can be a polyisocyanate of formula

R(NCO)ₙ,

wherein R is an aliphatic alkyl containing 2 to 18 carbon atoms, an aromatic alkyl containing 6 to 15 carbon atoms, or an araliphatic alkyl containing 8 to 15 carbon atoms and n is an integer from 2 to 4. Examples of polyisocyanates useful for preparing the isocyanate-terminated prepolymers of the present invention include, but are not limited to, ethylene diisocyanate, 1,4-tetramethylene diisocyanate, 1,6-hexamethylene diisocyanate (HDI), 1,2-dodecane diisocyanate, cyclobutane-1,3-diisocyanate, cyclohexane-1,3-diisocyanates, cyclohexane-1,4-diisocyanates, 1-isocyanato-3,3,5-trimethyl-5-isocyanatomethyl-cyclohexane, 2,4-hexahydrotoluene diisocyanates, 2,6-hexahydrotoluene diisocyanates, hexahydro-1,3-phenylene diisocyanate, hexahydro-1,4-phenylene diisocyanate, perhydro-2,4-diphenylmethane diisocyanate, perhydro-4,4′-diphenylmethane diisocyanate, 1,3-phenylene diisocyanate, 1,4-phenylene diisocyanate, 1,4-durol diisocyanate, 1,4-stilbene diisocyanate, 3,3′-dimethyl-4,4′-biphenylene diisocyanate, toluene 2,4-diisocyanates(TDI), toluene 2,6-diisocyanates (TDI), diphenylmethane-2,4′-diisocyanates (MDI), diphenylmethane-2,2′-diisocyanates (MDI), diphenylmethane-4,4′-diisocyanates (MDI), naphthylene-1,5-diisocyanate (NDI), their isomers, and mixtures thereof.

Polyisocyanates useful for preparing the isocyanate-terminated prepolymers of the present invention also includes any of the above polyisocyanates which have been modified to contain carbodiimide, allophanate, or isocyanurate structures. Such modified polyisocyanates include, but are not limited to, diphenylmethane diisocyanate, carbodiimide-modified diphenylmethane diisocyanate, their isomers, and mixtures thereof.

The polyol used to prepare the isocyanate-terminated prepolymers of the present invention can be one polyol or the mixture of polyols. The average molecular weight of these polyols is in the range of from 1000 to 10000 and the functionality of these polyols is in the range of from 1 to 5, preferably in the range of from 2 to 3. Examples of such polyols include, but are not limited to, polyester polyols, polyether polyols, polycarbonate polyols, and mixtures thereof.

The polyester polyols useful in preparing the isocyanate-terminated prepolymers of the present invention can be produced from the reaction of dicarboxylic acids or dicarboxylic acid anhydrides with polyhydric alcohols. Examples of dicarboxylic acids useful for this purpose include, but are not limited to, aliphatic carboxylic acids containing 2 to 12 carbon atoms, such as succinic acid, malonic acid, glutaric acid, adipic acid, suberic acid, azelaic acid, sebacic acid, decane-dicarboxylic acid, maleic acid, fumaric acid, phthalic acid, isophthalic acid, terephthalic acid, and mixtures thereof. Examples of dicarboxylic acid anhydrides useful for this purpose include, but are not limited to, phthalic anhydride, terachlorophthalic anhydride, maleic anhydride, and mixtures thereof. Examples of polyhydric alcohols useful for this purpose include, but are not limited to, ethanediol, diethylene glycol, 1,2-propanediol, 1,3-propanediol, dipropylene glycol, 1,3-methylpropanediol, 1,4-butanediol, 1,5-pentanediol, 1,6-hexanediol, neopentyl glycol, 1,10-decanediol, glycerol, trimethylol-propane, or mixtures thereof. These polyester polyols also include those prepared from lactones, including, but not limited to, ε-caprolactone.

The polyether polyols useful in preparing the isocyanate-terminated prepolymers of the present invention can be produced according to known processes, for example, by reaction of alkene oxides with polyhydric alcohol starters in the presence catalysts. Useful catalysts for this purpose include, but are not limited to, alkali hydroxides, alkali alkoxides, antimony pentachloride, boron fluoride etherate, and mixtures thereof. Useful alkene oxides for this purpose include, but are not limited to, tetrahydrofuran, ethylene oxide, 1,2-propylene oxide, 1,2-butylene oxide, 2,3-butylene oxide, styrene oxide, and mixtures thereof. Useful polyhydric alcohol starters for this purpose include, but are not limited to, polyhydric compounds such as water, ethylene glycol, 1,2-propanediols, 1,3-propanediols, 1,4-butanediol, diethylene glycol, trimethylol-propane, and mixtures thereof.

The polycarbonate polyols useful in preparing the isocyanate-terminated prepolymers of the present invention include, but are not limited to, polycarbonate diols. These polycarbonate diols can be produced by the reaction of diols with dialkyl or diaryl carbonates or phosgene. Useful diols for this purpose include, but are not limited to, 1,2-propanediol, 1,3-propanediol, 1,4-butanediol, 1,5-pentanediol, 1,6-hexanediol, diethylene glycol, trioxyethylene glycol, and mixtures thereof. Useful diaryl carbonates for this purpose include, but are not limited to, diphenyl carbonate.

Additional components used to prepare the isocyanate-terminated prepolymers of the present invention optionally include lactones, oxalates, or mixtures thereof. Useful lactones include, but are not limited to, γ-butyrolactone, γ-valerolactone, ε-caprolactone, αγ-dimethyl butyrolactone, βγ-dimethyl butyrolactone, γγ-dimethyl butyrolactone, α-ethyl-γ-methyl butyrolactone, and mixtures thereof. Useful oxalates include, but are not limited to, dimethyl oxalate, diethyl oxalate, and dibutyl oxalate, and mixtures thereof. Lactone, oxalate, or mixtures thereof can be added in an amount in the range of from 1 to 15 weight %, preferably in an amount in the range of from 3 to 7 weight %, based on 100 weight % of the isocyanate-terminated prepolymer and polyols.

The NCO content of the isocyanate-terminated prepolymers of the present invention is in the range of from 13 to 33 weight %, preferably in the range of from 13 to 24 weight %, most preferably in the range of from 15 to 20 weight %, based on 100% by weight of the isocyanate-terminated prepolymer.

The isocyanate-terminated prepolymers of the present invention can be used to prepare polyurethanes, polyureas, polyurethane elastomers, and moulded polyurethanes. When the NCO content of the isocyanate-terminated prepolymer is in the range of from 13 to 25 weight %, the prepolymer is suitable to be used to prepare polyurethane elastomers. When the NCO content of the isocyanate-terminated prepolymer is in the range of from 20 to 33 weight %, the prepolymer is suitable to be used to prepare moulded polyurethanes.

The above polyurethanes and polyureas comprise the reaction product of the isocyanate-terminated prepolymers of the present invention with polyols and chain extenders.

Polyols useful for preparing the polyurethanes and polyureas of the present invention include, but are not limited to, those listed above.

Chain extenders useful for preparing the polyurethanes and polyureas of the present invention typically include, but are not limited to, active hydrogen atom containing compounds having molecular weights of less than 800, preferably in the range of from 18 to 400. Examples of such active hydrogen atom containing compounds include, but are not limited to, alkanediols, such as ethanediol, 1,4-butanediol, 1,6-hexanediol, 1,7-heptanediol, 1,8-octanediol, 1,9-nonanediol, and 1,10-decanediol, dialkylene glycols, such as diethylene glycol and dipropylene glycol, polyalkylene polyols, such as polyoxyalkylene glycols, and mixtures thereof. Useful active hydrogen atom containing compounds also include, but are not limited to, branched chain and unsaturated alkanediols, such as 1,2-propanediol, 2-methyl-1,3-propanediol, 2,2-dimethyl-1,3-propanediol, 2-butyl-2-ethyl-1,3-propanediol, 2-butene-1,4-diol, and 2-butyne-1,4-diol, alkanolamines, such as ethanolamine, 2-aminopropanol, 3-amino-2,2-dimethylpropanol, and N-alkyldialkanolamines, such as N-methyl-diethanolamines and N-ethyl-diethanolamines, and mixtures thereof. Useful active hydrogen atom containing compounds also includes, but are not limited to aliphatic amines and aromatic amines, such as 1,2-ethylenediamine, 1,3-propylenediamine, 1,4-butylenediamine, 1,6-hexamethylenediamine, isophoronediamine, 1,4-cyclohexamethylenediamine, N,N′-diethyl-phenylenediamine, 2,4-diaminotoluene, 2,6-diaminotoluene, and mixtures thereof.

The polyurethanes and polyureas prepared from the isocyanate-terminated prepolymers of the present invention can also be prepared in the presence of blowing agents, catalysts, and surfactants.

Blowing agents useful in the present invention include, but are not limited to, water, halohydrocarbons, hydrocarbons, and gases. Examples of halohydrocarbons useful as blowing agents in the present invention include, but are not limited to, monochlorodifuloromethane, dichloromonofluoromethane, dichlorofluoromethane, trichlorofluromethane, and mixtures thereof. Examples of hydrocarbons useful as blowing agents in the present invention include, but are not limited to, butane, pentane, cyclopentane, hexane, cyclohexane, heptane, and mixtures thereof Examples of gases useful as blowing agents in the present invention include, but are not limited to, air, CO₂, N₂, and mixtures thereof.

Catalysts useful in the present invention include, but are not limited to, amines, organo-metallic compounds, and mixtures thereof. Examples of amines useful as catalysts in the present invention include, but are not limited to, triethylamine, tributylamine, N-methylmorpholine, N-ethylmorpholine, N,N,N′,N′-tetramethyl-ethylenediamine, pentamethyldiethylene-triamine, N,N-methylbenzylamine, N,N-dimethylbenzylamine, and mixtures thereof. Examples of organo-metallic compounds useful as catalysts in the present invention include, but are not limited to, organotin compounds, such as tin (II) acetate, tin (II) octoate, tin (II) ethylhexonate, tin (II) laurate, dibutyltin oxide, dibutyltin dichloride, dibutyltin diacetate, dibutyltin dilaurate, dibutyltin maleate, dioctyltin diacetate, and mixtures thereof. The amount of catalyst used is in the range of from 0.001 to 10 weight %, based on 100 weight % of the polyols used in the reaction system (not only including the polyols used as reaction composition, but also including the polyols used as chain extenders and the polyols used in other composition).

Surfactants useful in the present invention include, but are not limited to, polyoxyalkylene derivatives of siloxane. The amount of the surfactant used is in the range of from 0.01 to 5 weight %, based on 100 weight % of the polyols used in the reaction system (not only including the polyols used as reaction composition, but also including the polyols used as chain extenders and the polyols used in other composition).

Polyurethane elastomers prepared from the isocyanate-terminated prepolymers of the present invention can be used in the manufacture of shoes, in particular, in the manufacture of shoe soles and/or shoe uppers.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

### Preparation of the Isocyanate-Terminated Prepolymer

Normally, at low temperatures, the liquid stability of the isocyanate-terminated prepolymer can be characterized by its freezing point. In this invention, the freezing point was tested according to the following method:
1) A glass tube with prepolymer sample sealed inside was put into an incubator at a predetermined temperature X° C. After the sample reached the predetermined temperature for a period of time (for example, 10 to 15 hours), the sample was checked as to whether any concretion or crystals appeared;
2) If no concretion or crystals appeared, the predetermined temperature X° C. was decreased, the test process described in step 1) was repeated. If concretion or crystals appeared, the predetermined temperature X° C. was increased and the test process described in step 1) was repeated. X° C. was the freezing point of the isocyanate-terminated prepolymer if concretion or crystals appeared at a predetermined temperature X° C., but did not appear at a predetermined temperature (X+1)° C.

According to above-mentioned method, the error of the freezing point of the isocyanate-terminated prepolymer was ±1° C.

The materials used in the following experiments are as follows:

Bayflex 2003E: polyester polyols, average molecular weight of 2000, functionality of 2, available from Bayer;

Arcol Polyol 1021: polyether polyols, average molecular weight of 2000, functionality of 2, available from Bayer;

Dabco EG: tertiary amine catalyst, available from Air Products;

DC 193: silane surfactants, available from Air Products.

### EXAMPLES 1-10

4,4′-MDI (56 wt. %) and Bayflex 2003E (38 wt. %) were added into a reactor and reacted at 70° C. for 2 hours. The temperature of the reactor was reduced to 65° C. A carbodiimide-modified MDI (6 wt. %) was added into the reactor, stirred for 30 minutes, and then a mixture A was obtained. The temperature of the mixture A was reduced to room temperature. Propylene carbonate was added into the mixture A, wherein the amount of the propylene carbonate was listed in Table 1. The mixture A was stirred for 30 minutes and then a prepolymer was obtained. The test results of the prepolymer are listed in Table 1.

### EXAMPLE 11

4,4′-MDI (43 wt. %) and Bayflex 2003E (51 wt. %) were added into a reactor and reacted at 70° C. for 2 hours. The temperature of the reactor was reduced to 65° C. A carbodiimide-modified MDI (6 wt. %) was added into the reactor, stirred for 30 minutes, and then a mixture B was obtained. The temperature of the mixture B was reduced to room temperature. Propylene carbonate (5 wt. %) was added into the mixture B. The mixture B was stirred for 30 minutes and then a prepolymer was obtained. The test results of the prepolymer were listed in Table 1.

TABLE 1

| Isocyanate- | | | | | | | | | | | |
|---|---|---|---|---|---|---|---|---|---|---|---|
| terminated | | | | | | | | | | | |
| prepolymer | (1)* | (2) | (3) | (4) | (5) | (6) | (7) | (8) | (9) | (10) | (11) |
| Mixture (wt. %) | 100 | 100 | 100 | 100 | 100 | 100 | 100 | 100 | 100 | 100 | 100 |
| Propylene | — | 1 | 3 | 5 | 3.75 | 2.5 | 1.25 | 2.5 | 7 | 15 | 5 |
| carbonate (wt. %) | | | | | | | | | | | |
| γ-butyrolactone | — | — | — | — | 1.25 | 2.5 | 3.75 | — | — | — | — |
| (wt. %) | | | | | | | | | | | |
| Diethyl oxalate | — | — | — | — | — | — | — | 2.5 | — | — | — |
| (wt. %) | | | | | | | | | | | |
| NCO (wt. %) | 19.3 | 19.1 | 18.7 | 18.4 | 18.4 | 18.4 | 18.4 | 18.4 | 18.0 | 16.8 | 12.7 |
| Freezing point | 12 | 11 | 9 | 6 | 6 | 6 | 5 | 5 | 4 | −3 | <0 |
| (±1° C.) | | | | | | | | | | | |

*Comparative Examples

Table 1 demonstrates that the freezing point of isocyanate-terminated prepolymers can be significantly reduced by adding a suitable amount of propylene carbonate or adding a suitable amount of propylene carbonate with lactone and/or oxalate.

### EXAMPLES 12 and 13

4,4′-MDI (75 wt. %) and Bayflex 2003E (38 wt. %) were added into a reactor and reacted at 70° C. for 2 hours. The temperature of the reactor was reduced to 65° C. A carbodiimide-modified MDI (6 wt. %) was added into the reactor, stirred for 30 minutes, and then a mixture C was obtained. The temperature of the mixture C was reduced to room temperature. Propylene carbonate (5 wt. %) was added into the mixture C. The mixture C was stirred for 30 minutes and then a prepolymer was obtained. The test results of the prepolymer were listed in Table 2.

### EXAMPLES 14 and 15

4,4′-MDI (66 wt. %), Arcol Polyol 1021 (22 wt. %) and tripropylene glycol (7 wt. %) were added into a reactor and reacted at 70° C. for 2 hours. The temperature of the reactor was reduced to 65° C. A carbodiimide-modified MDI (5 wt. %) was added into the reactor, stirred for 30 minutes, and then a mixture D was obtained. The temperature of the mixture D was reduced to room temperature. Propylene carbonate was added into the mixture D, wherein the amount of the propylene carbonate is listed in Table 2. The mixture D was stirred for 30 minutes and then a prepolymer was obtained. The test results of the prepolymer were listed in Table 2.

TABLE 2

| Isocyanate-terminated | | | | |
|---|---|---|---|---|
| prepolymer | (12)** | (13) | (14)** | (15) |
| Mixture (wt. %) | Mixture C | Mixture C | Mixture D | Mixture D |
| | 100 | 100 | 100 | 100 |
| Propylene carbonate | — | 5 | — | 5 |
| (wt. %) | | | | |
| NCO (wt. %) | 25.0 | 23.8 | 20.1 | 19.1 |
| Freezing point (±1° C.) | 20 | 15 | 8 | 2 |

**Comparative Examples

Table 2 demonstrates that when NCO contents are similar, the freezing point of isocyanate-terminated prepolymer can be significantly reduced by adding a suitable amount of propylene carbonate.

### Preparation of the Polyurethane Elastomer

The polyurethane elastomer was a reaction product of the isocyanate-terminated prepolymers according to the present invention, polyols, and chain extenders. Furthermore, blowing agents, catalysts and surfactants can be optionally added as reaction components. The reaction temperature was 20-80° C., preferably 30-60° C. When the reaction was finished, the mixture was put into a closed mould. The demould time was from 2 to 15 minutes.

### EXAMPLES 16-18

The polyurethane elastomer was prepared from the isocyanate-terminated prepolymers of Examples 4, 6, and 8. According to Table 3, the reaction components were mixed by means of mechanical stirring at 45° C. for preparing the polyurethane elastomer. The mixture was poured into an aluminum mould, and demoulded after the mould was closed. The demould time was 3 minutes. The test results of the polyurethane elastomer were listed in table 4.

TABLE 3

| | Isocyanate-terminated prepolymer |
|---|---|
| Isocyanate-terminated prepolymer | (4)□(6)□(8) |
| Bayflex 2003 E | 100 wt. % |
| Glycol | 7 wt. % |
| Water | 0.4 wt. % |
| Amine catalyst Dabco EG | 1.5 wt. % |
| Surfactant DC193 | 0.4 wt. % |

TABLE 4

| | Example | | |
|---|---|---|---|
| | 16 | 17 | 18 |
| Isocyanate-terminated prepolymer | (4) | (6) | (8) |
| Density (kg/m³) | 0.55 | 0.55 | 0.55 |
| Tensile strength (MPa) | 4.1 | 2.9 | 3.2 |
| Tear strength (kN/m) | 21.3 | 19.7 | 19.2 |
| Split tear strength (kN/m) | 7.4 | 7.6 | 6.5 |
| Elongation (%) | 482 | 473 | 451 |
| Ross flex (mm) (room temp, 40k cycles) | 0.1 | 0.2 | 0.1 |
| Hardness (Shore A) | 43 | 43 | 43 |
| Abrasion (mm³) (5N half distance) | 160 | 198 | 201 |

Table 3 and Table 4 demonstrate that the polyurethane elastomer prepared by using of the isocyanate-terminated prepolymer provided by this invention possessed similar physical properties as the polyurethane elastomer in prior art.

1. A isocyanate-terminated prepolymer prepared from
A) a polyisocyanate;
B) a polyol; and
C) a cyclic alkylene carbonate of formula: 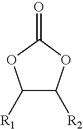
wherein
R₁ and R₂ are independently selected from the group consisting of hydrogen, linear alkyl, branched alkyl, aralkyl, naphthenic base, and aryl; and
the NCO content of said isocyanate-terminated prepolymer is in the range of from 13 to 33% by weight, based on 100% by weight of said isocyanate-terminated prepolymer.

2. The isocyanate-terminated prepolymer of claim 1, wherein said polyisocyanate is of formula:
R(NCO)ₙ
wherein
R is an aliphatic alkyl containing 2 to 18 carbon atoms, an aryl containing from 6 to 15 carbon atoms, or an araliphatic alkyl containing from 8 to 15 carbon atoms; and
n is an integer from 2 to 4.

3. The isocyanate-terminated prepolymer of claim 2, wherein said polyisocyanate is selected from the group consisting of ethylene diisocyanate; 1,4-tetramethylene diisocyanate; 1,6-hexamethylene diisocyanate; 1,2-dodecane diisocyanate; cyclobutane-1,3-diisocyanate; cyclohexane-1,3-diisocyanates; cyclohexane-1,4-diisocyanates; 1-isocyanato-3,3,5-trimethyl-5-isocyanatomethyl-cyclohexane; 2,4-hexahydrotoluene diisocyanates; 2,6-hexahydrotoluene diisocyanates; hexahydro-1,3-phenylene diisocyanate; hexahydro-1,4-phenylene diisocyanate; perhydro-2,4-diphenylmethane diisocyanate; perhydro-4,4′-diphenylmethane diisocyanate; 1,3-phenylene diisocyanate; 1,4-phenylene diisocyanate; 1,4-durol diisocyanate; 1,4-stilbene diisocyanate; 3,3′-dimethyl-4,4′-biphenylene diisocyanate, toluene 2,4-diisocyanates, toluene 2,6-diisocyanates, diphenylmethane-2,4′-diisocyanates, diphenylmethane-2,2′-diisocyanates, diphenylmethane-4,4′-diisocyanates, naphthylene-1,5-diisocyanate, and isomers and mixtures thereof.

4. The isocyanate-terminated prepolymer of claim 1, wherein said polyol is a polyester polyol, a polyether polyol, a polycarbonate polyol, or a mixture thereof.

5. The isocyanate-terminated prepolymer of claim 4, wherein said polyester polyol is prepared from the reaction of a dicarboxylic acid selected from the group consisting of succinic acid, malonic acid, glutaric acid, adipic acid, suberic acid, azelaic acid, sebacic acid, decane-dicarboxylic acid, maleic acid, fumaric acid, phthalic acid, isophthalic acid, terephthalic acid, and mixtures thereof or a dicarboxylic acid anhydride selected from the group consisting of phthalic anhydride, terachlorophthalic anhydride, maleic anhydride, and mixtures thereof with a polyhydric alcohol selected from the group consisting of ethanediol, diethylene glycol, 1,2-propanediol, 1,3-propanediol, dipropylene glycol, 1,3-methylpropanediol, 1,4-butanediol, 1,5-pentanediol, 1,6-hexanediol, neopentyl glycol, 1,10-decanediol, glycerol, trimethylol-propane, and mixtures thereof.

6. The isocyanate-terminated prepolymer of claim 4, wherein said polyester polyol is prepared using ε-caprolactone.

7. The isocyanate-terminated prepolymer of claim 4, wherein said polyether polyol is prepared from the reaction of an alkene oxide selected from the group consisting of tetrahydrofuran, ethylene oxide, 1,2-propylene oxide, 1,2-butylene oxide, 2,3-butylene oxide, styrene oxide, and mixtures thereof with a polyhydric alcohol starter selected from the group consisting of water, ethylene glycol, 1,2-propanediols, 1,3-propanediols, 1,4-butanediol, diethylene glycol, trimethylol-propane, and mixtures thereof.

8. The isocyanate-terminated prepolymer of claim 4, wherein said polycarbonate polyol is a polycarbonate diol prepared from the reaction of a diol selected from the group consisting of 1,2-propanediol, 1,3-propanediol, 1,4-butanediol, 1,5-pentanediol, 1,6-hexanediol, diethylene glycol, trioxyethylene glycol, and mixtures thereof with diphenyl carbonate or phosgene.

9. The isocyanate-terminated prepolymer of claim 1, wherein said prepolymer is further prepared from a lactone, an oxalate, or a mixture thereof.

10. The isocyanate-terminated prepolymer of claim 9, wherein said lactone is selected from the group consisting of γ-butyrolactone, γ-valerolactone, ε-caprolactone, αγ-dimethyl butyrolactone, βγ-dimethyl butyrolactone, γγ-dimethyl butyrolactone, and α-ethyl-γ-methyl butyrolactone, and said oxalate is selected from the group consisting of dimethyl oxalate, diethyl oxalate, and dibutyl oxalate.

11. The isocyanate-terminated prepolymer of claim 1, wherein the NCO content of said isocyanate-terminated prepolymer is in the range of from 13 to 24% by weight, based on 100% by weight of said isocyanate-terminated prepolymer.

12. The isocyanate-terminated prepolymer of claim 11, wherein the NCO content of said isocyanate-terminated prepolymer is in the range of from 15 to 20% by weight, based on 100% by weight of said isocyanate-terminated prepolymer.

13. A process for preparing the isocyanate-terminated prepolymer of claim 1 comprising reacting
A) a polyisocyanate;
B) a polyol; and
C) a cyclic alkylene carbonate of formula: 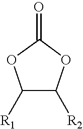
wherein
R₁ and R₂ are independently selected from the group consisting of hydrogen, linear alkyl, branched alkyl, aralkyl, naphthenic base, and aryl
wherein said cyclic alkylene carbonate is added to the reaction with A) and B), during the reaction of A) and B), or after the reaction of A) and B); and
wherein the NCO content of said isocyanate-terminated prepolymer is in the range of from 13 to 33% by weight, based on 100% by weight of said isocyanate-terminated prepolymer.

14. A polyurethane or polyurea or polyurethane elastomer or polyurethane moulding comprising the isocyanate-terminated prepolymer of claim 1.

15. A shoe sole comprising the isocyanate-terminated prepolymer of claim 1.
